(19) United States
(12) Patent Application Publication
(10) Pub. No.: US 2024/0307080 A1
(43) Pub. Date: Sep. 19, 2024

(54) SYSTEMS AND METHODS FOR CAVITATION MAPPING WITH SPATIAL-TEMPORAL PARALLEL PROCESSING

(71) Applicant: THE TRUSTEES OF COLUMBIA UNIVERSITY IN THE CITY OF NEW YORK, New York, NY (US)

(72) Inventors: Elisa KONOFAGOU, New York, NY (US); Sua BAE, New York, NY (US)

(73) Assignee: THE TRUSTEES OF COLUMBIA UNIVERSITY IN THE CITY OF NEW YORK, New York, NY (US)

(21) Appl. No.: 18/670,828

(22) Filed: May 22, 2024

Related U.S. Application Data
(63) Continuation of application No. PCT/US22/50572, filed on Nov. 21, 2022.
(60) Provisional application No. 63/282,000, filed on Nov. 22, 2021.

(51) Int. Cl. A61B 17/22; A61B 8/00; A61B 8/08; A61B 17/00

(52) U.S. Cl. CPC A61B 17/22004; A61B 8/0808; A61B 8/4488; A61B 8/481; A61B 2017/00106; A61B 2017/22008; A61B 2017/22027

(57) ABSTRACT

The present subject matter relates to techniques for passive acoustic mapping. The disclosed system can include a focused ultrasound (FUS) transducer, a diagnostic phase array transducer, and a processor. The diagnostic phase array transducer can be configured to receive a cavitation signal induced from cavitation. The processor can be configured to generate a cavitation map based on a spatio-temporal cavitation intensity. The spatio-temporal cavitation intensity can be calculated using a spatial-temporal parallel programming. The spatial-temporal parallel programming can be performed by creating a thread for each pixel of the spatial-temporal map, calculating the spatio-temporal cavitation intensity at a location and a time point in each thread, and creating a cavitation map by integrating the spatial-temporal cavitation intensity over temporal pixels.

### CROSS-REFERENCE TO RELATED APPLICATION

This application is a continuation of International Patent Application No. PCT/US2022/050572, which was filed on Nov. 21, 2022, which claims priority to U.S. Provisional Patent Application No. 63/282,000, which was filed on Nov. 22, 2021, the entire contents of which are incorporated by reference herein.

### GRANT INFORMATION

This invention was made with government support under grant numbers AG038961 and EB009041 awarded by the National Institutes of Health. The government has certain rights in the invention.

### BACKGROUND

Focused ultrasound (FUS) can induce cavitation activities of microbubbles and open the blood-brain barrier (BBB) of the targeted region transiently for localized drug delivery into the brain parenchyma or for stimulating the immune response. Since the BBB opening with microbubble-mediated FUS can be based on the linear and nonlinear oscillations of the bubbles, called cavitation, the acoustic emissions from the bubbles during the sonication can be passively detected and analyzed for treatment monitoring. Cavitation signals from the bubbles can be detected by certain MR-guided FUS. In the portable neuronavigation-guided system, cavitation signals can be detected by a single-element PCD placed at the central opening of the FUS transducer.

Spatial information on microbubble cavitation can be important for the safe and efficient treatment since microbubble cavitation can occur outside of the focal region in the brain depending on cerebral vascularity and the brain tissue structures (gyri and sulci and boundaries of ventricles).

Cavitation mapping with a linear array transducer can have a poor spatial resolution because of the small receiving aperture and the uncertainty of the time of cavitation occurrence.

Therefore, there is a need for improved techniques for cavitation mapping.

### SUMMARY

The disclosed subject matter provides techniques for passive acoustic mapping.

An example system includes a focused ultrasound (FUS) transducer, a diagnostic phase array transducer, and a processor. The diagnostic phase array transducer can be configured to receive a cavitation signal induced from cavitation. The processor can be configured to generate a cavitation map based on a spatio-temporal cavitation intensity. The spatio-temporal cavitation intensity can be calculated using a spatial-temporal parallel programming. The spatial-temporal parallel programming can be performed by creating a thread for each pixel of the spatial-temporal map, calculating the spatio-temporal cavitation intensity at a location and a time point in each thread, and creating a cavitation map by integrating the spatial-temporal cavitation intensity over temporal pixels.

In certain embodiments, a length of the FUS bust can be less than 10 milliseconds.

In certain embodiments, the system further can further include microbubbles. The microbubbles can be configured to induce the cavitation. In non-limiting embodiments, the system can include a neuronavigation system configured to position the FUS transducer at a target area.

In certain embodiments, the FUS transducer can be a single-element FUS transducer. In non-limiting embodiments, the FUS transducer can have a center frequency about 0.25 MHz.

In certain embodiments, the diagnostic phase array transducer can have a plurality of elements. In non-limiting embodiments, the diagnostic phase array transducer has a center frequency about 2.5 MHz. In non-limiting embodiments, the diagnostic phase array transducer can be inserted into a central opening of the FUS transducer. In non-limiting embodiments, the diagnostic phase array transducer can be configured to acquire the cavitation signal at a sample rate of about 10 MHz.

The disclosed subject matter also provides methods for passive acoustic mapping. An example method can include applying a focused ultrasound to induce a cavitation signal, receiving the cavitation signal, and calculating a spatio-temporal cavitation intensity using a spatial-temporal parallel programming. The spatial-temporal parallel programming can be performed by creating a thread for each pixel of the spatial-temporal map, calculating the spatio-temporal cavitation intensity at a location and a time point in each thread, and creating a cavitation map by integrating the spatio-temporal cavitation intensity over temporal pixels. The cavitation signal can be a radio frequency signal.

In certain embodiments, a length of the FUS bust is less than 10 milliseconds.

In certain embodiments, the method can include introducing microbubbles to a target area, wherein the microbubbles are configured to induce the cavitation. In non-limiting embodiments, the target area is a blood-brain barrier.

In certain embodiments, the method can further include positioning a FUS transducer to a target area using a neuronavigation system. In non-limiting embodiments, the FUS transducer is configured to apply a focused ultrasound to the target. In non-limiting embodiments, the FUS transducer has a center frequency about 0.25 MHz.

In certain embodiments, the method can include modifying a parameter of the FUS transducer. In non-limiting embodiments, the parameter can be selected from a center frequency, an outer diameter, an inner diameter, a radius of curvature, and a combination thereof.

The disclosed subject matter will be further described below.

### BRIEF DESCRIPTION OF THE DRAWINGS

FIGS. 1A-1C provide diagrams and images showing an example system and method for the reconstruction of a cavitation map in accordance with the disclosed subject matter.

FIG. 2 provides a graph showing example processing times for reconstruction of a single frame of cavitation map in accordance with the disclosed subject matter.

FIGS. 3A-3H provide images showing an example of spatial resolution evaluation in accordance with the disclosed subject matter.

FIGS. 4A-4C provide example cavitation maps reconstructed by using (4A) TEA, (4B)ERCB, and (4C) CF-PAM in accordance with the disclosed subject matter.

FIGS. 5A-5F provide images and graphs showing the example of In vivo cavitation mapping techniques in accordance with the disclosed subject matter.

FIG. 6 provides a graph showing processing times with the various number of integration time samples in accordance with the disclosed subject matter.

It is to be understood that both the foregoing general description and the following detailed description are exemplary and are intended to provide further explanation of the disclosed subject matter.

### DETAILED DESCRIPTION

The disclosed subject matter provides multilateral techniques for cavitation mapping. The disclosed subject matter provides systems and methods for passive acoustic mapping through focused ultrasound (FUS).

Unless otherwise defined, all technical and scientific terms used herein have the same meaning as commonly understood by one of ordinary skill in the art. In case of conflict, the present document, including definitions, will control.

The terms “comprise(s),” “include(s),” “having,” “has,” “can,” “contain(s),” and variants thereof, as used herein, are intended to be open-ended transitional phrases, terms, or words that do not preclude additional acts or structures. The singular forms “a,” “an,” and “the” include plural references unless the context clearly dictates otherwise. The present disclosure also contemplates other embodiments “comprising,” “consisting of,” and “consisting essentially of,” the embodiments or elements presented herein, whether explicitly set forth or not.

As used herein, the term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, up to 10%, up to 5%, and up to 1% of a given value. Alternatively, e.g., with respect to biological systems or processes, the term can mean within an order of magnitude, within 5-fold, and within 2-fold, of a value.

The term “coupled,” as used herein, refers to the connection of a device component to another device component by methods known in the art.

As used herein, the term “subject” includes any human or nonhuman animal. The term “nonhuman animal” includes, but is not limited to, all vertebrates, e.g., mammals and non-mammals, such as nonhuman primates, dogs, cats, sheep, horses, cows, chickens, amphibians, reptiles, etc.

As used herein, “treatment” or “treating” refers to inhibiting the progression of a disease or disorder, or delaying the onset of a disease or disorder, whether physically, e.g., stabilization of a discernible symptom, physiologically, e.g., stabilization of a physical parameter, or both. As used herein, the terms “treatment,” “treating,” and the like refer to obtaining a desired pharmacologic and/or physiologic effect. The effect can be prophylactic in terms of completely or partially preventing a disease or condition or a symptom thereof and/or can be therapeutic in terms of a partial or complete cure for a disease or disorder and/or adverse effect attributable to the disease or disorder. “Treatment,” as used herein, covers any treatment of a disease or disorder in an animal or mammal, such as a human, and includes: decreasing the risk of death due to the disease, preventing the disease or disorder from occurring in a subject which can be predisposed to the disease but has not yet been diagnosed as having it; inhibiting the disease or disorder, i.e., arresting its development (e.g., reducing the rate of disease progression); and relieving the disease, i.e., causing regression of the disease.

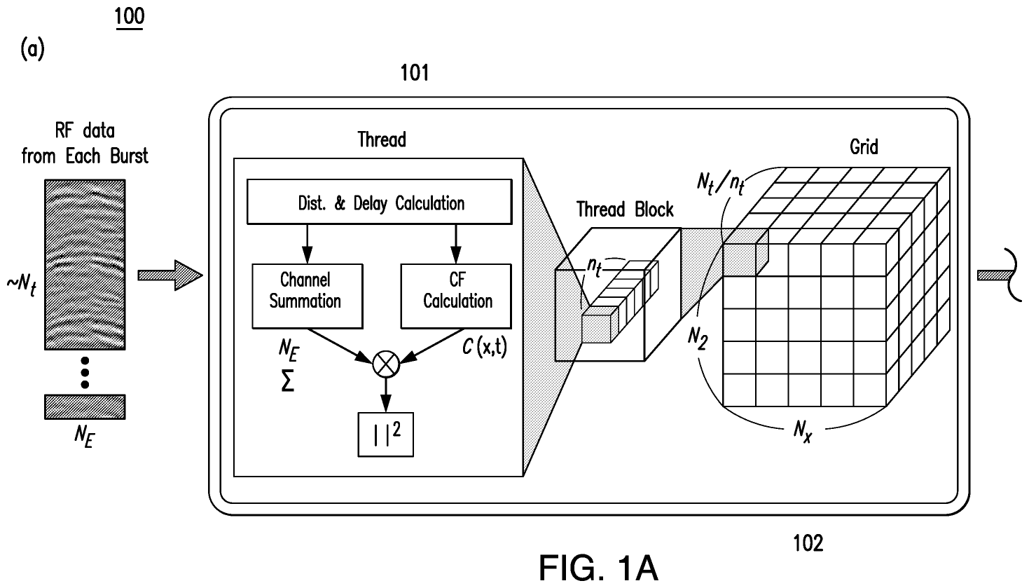

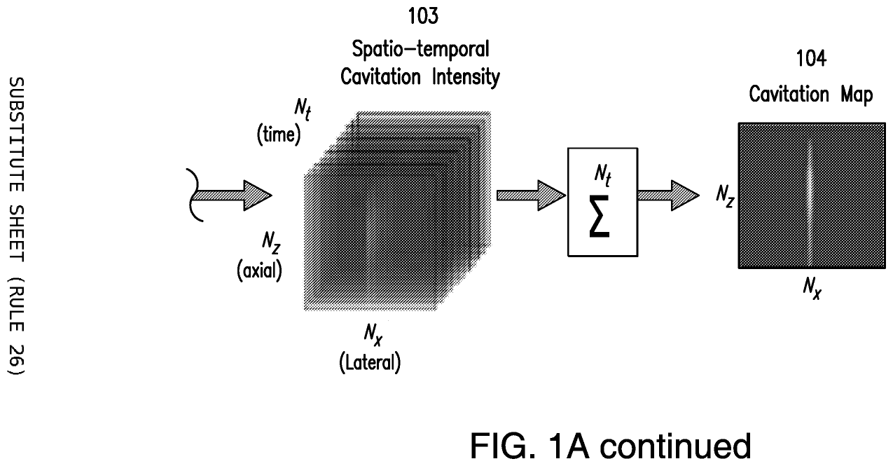

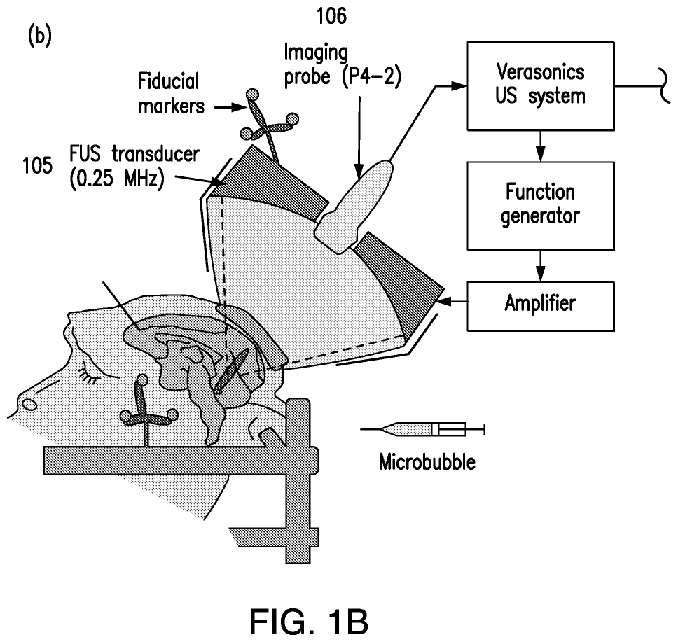

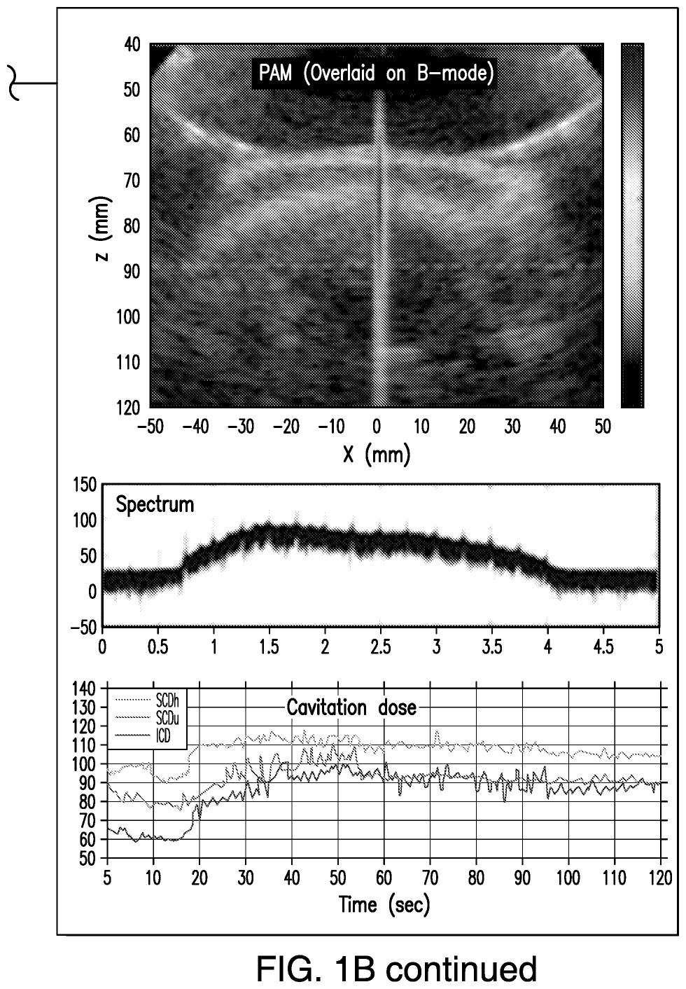

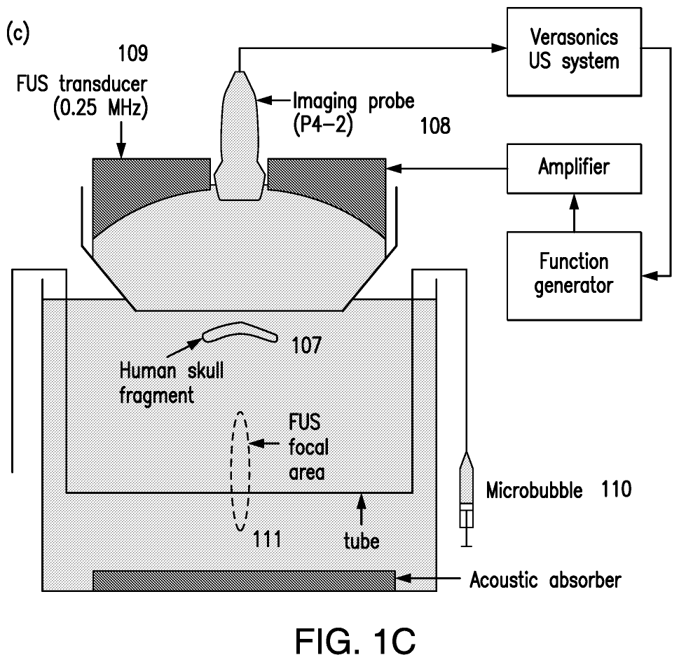

In certain embodiments, the disclosed subject matter provides a system for passive acoustic mapping. As shown in FIGS. 1A-1C, an example system 100 can include a focused ultrasound (FUS) transducer, a diagnostic phase array transducer, and a processor.

In non-limiting embodiments, the FUS transducer can be a single element that can be configured to apply a focused ultrasound to a target region. For example, the FUS transducer can be a single-element FUS transducer configured to induce FUS for opening target tissue. For example, the single-element transducer can generate an acoustic radiation force and induce cavitation at the target tissue. In some embodiments, the transducer can be connected to a function generator and have a predetermined ultrasound parameter to induce cavitation, open the target tissue, and/or activate microbubbles. In non-limiting embodiments, the parameters can be modified or adjusted depending on a target tissue, a subject, or a type of microbubble.

In certain embodiments, the predetermined ultrasound parameter of the FUS transducer can include a center frequency, a pressure, an outer diameter, an inner diameter, a radius of curvature, and a combination thereof. For example, the center frequency can range from about 20 kilohertz (kHz) to about 1 megahertz (MHz). In non-limiting embodiments, the center frequency can range from about 0.1 MHz to about 5 MHz, from about 0.1 MHz to about 4 MHz, from about 0.1 MHz to about 3 MHz, from about 0.1 MHz to about 2 MHz, from about 0.1 MHz to about 1 MHz, from about 0.1 MHz to about 0.5 MHz, from about 0.1 MHz to about 0.35 MHz, from about 0.2 MHz to about 0.35 MHZ, or from about 0.2 MHz to about 0.25 MHz. In non-limiting embodiments, the center frequency of the ultrasonic pulse that can be transmitted by the transducer can be up to about 0.2, about 0.25, about 0.35 MHZ, about 1 MHZ, about 2 MHZ, about 3 MHz, about 4 MHz, or about 5 MHz. In non-limiting embodiments, the pulse length can range from about 5 us to about 100 ms. In some embodiments, the pulse repetition frequency can range from about 1 Hz to about 5 kHz.

In certain embodiments, the predetermined ultrasound parameter of the FUS transducer can include the outer diameter, inner diameter, and radius curvature of the disclosed element transducer. The outer diameter of the single-element transducer can range from about 30 millimeters (mm) to about 200 mm, from about 30 mm to about 150 mm, from about 30 mm to about 110 mm, from about 40 mm to about 110 mm, from about 50 mm to about 110 mm, or from about 60 mm to about 110 mm. In non-limiting embodiments, the outer diameter of the single-element transducer can be about 60 or 110 mm. In some embodiments, the inner diameter of the single-element transducer can range from about 10 (mm) to about 60 mm, from about 10 mm to about 50 mm, from about 20 mm to about 50 mm, or from about 30 mm to about 50. In non-limiting embodiments, the inner diameter of the single-element transducer can be about 44 mm. In some embodiments, the radius of curvature can range from about 30 millimeters (mm) to about 200 mm, from about 30 mm to about 150 mm, from about 30 mm to about 110 mm, from about 40 mm to about 110 mm, from about 50 mm to about 110 mm, from about 60 mm to about 110 mm, or from about 70 mm to about 110 mm. In non-limiting embodiments, the radius curvature can be about 70, 76, or 110 mm.

In certain embodiments, the predetermined ultrasound parameter of the FUS transducer can include a mechanical index, pulse length, pulse repetition frequency, peak-negative pressure, and sonication duration. For example, the pulse length can range from about 0.001 milliseconds (ms) to about 100 ms, from about 0.001 ms to about 90 ms, from 0.001 ms to about 80 ms, from 0.001 ms to about 70 ms, from 0.001 ms to about 60 ms, from 0.001 ms to about 50 ms, from 0.001 ms to about 40 ms, from 0.001 ms to about 30 ms, from 0.001 ms to about 20 ms, or from 0.001 ms to about 10 ms. In non-limiting embodiments, the pulse length can be about 10 ms. The pulse length can also range from about 1 cycle to about 5000 cycles, from about 1 cycle to about 4000 cycles, from about 1 cycle to about 10,000 cycles, from about 1 cycle to about 5000 cycles, from about 1 cycle to about 4000 cycles, from about 1 cycle to about 3000 cycles, from about 1 cycle to about 2500 cycles, from about 500 cycles to about 2500 cycles, from about 1000 cycles to about 2500 cycles, from about 1500 cycles to about 2500 cycles, or from about 2000 cycles to about 2500 cycles. The pulse repetition frequency can range from about 0.1 Hz to about 10 kHz, from about 0.1 Hz to about 9 kHz, from about 0.1 Hz to about 8 kHz, from about 0.1 Hz to about 7 kHz, from about 0.1 Hz to about 6 kHz, from about 0.1 Hz to about 5 kHz, from about 0.1 Hz to about 4 kHz, from about 0.1 Hz to about 3 kHz, or from about 0.1 Hz to about 2 kHz. In non-limiting embodiments, the pulse repetition frequency can be about 2 Hz.

In certain embodiments, the sonication duration can range from about 0.1 minutes to about 5 minutes, from about 0.1 minutes to about 4 minutes, from about 0.1 minutes to about 3 minutes, from about 0.1 minutes to about 2 minutes, from about 0.5 minutes to about 2 minutes, or from about 1 minute to about 2 minutes. In non-limiting embodiments, the sonication duration can be about 2 minutes. In non-limiting embodiments, the FUS burst time can range from about 1 millisecond to about 1000 milliseconds, from about 1 millisecond to about 700 milliseconds, from about 1 millisecond to about 500 milliseconds, from about 1 millisecond to about 300 milliseconds, from about 1 millisecond to about 200 milliseconds, from about 1 millisecond to about 100 milliseconds, from about 1 millisecond to about 50 milliseconds, from about 1 millisecond to about 10 milliseconds, or from about 1 millisecond to about 5 milliseconds. In non-limiting embodiments, the FUS burst time can be the pulse length.

In certain embodiments, the peak-negative pressure parameter of the FUS transducer can range from about 0.05 MPa to about 10 MPa, from about 0.1 MPa to about 9 MPa, from about 0.1 MPa to about 8 MPa, from about 0.1 MPa to about 7 MPa, from about 0.1 MPa to about 6 MPa, from about 0.1 MPa to about 5 MPa, from about 0.1 MPa to about 4 MPa, from about 0.1 MPa to about 3 MPa, from about 0.1 MPa to about 2 MPa, from about 0.1 MPa to about 1 MPa, from about 0.1 MPa to about 0.5 MPa, from about 0.1 MPa to about 0.4 MPa, from about 0.1 MPa to about 0.3 MPa, or from about 0.1 MPa to about 0.2 MPa. In non-limiting embodiments, the peak-negative pressure can be about 0.2 MPa.

In certain embodiments, the disclosed system can include a diagnostic phase array transducer. In non-limiting embodiments, the diagnostic phase array can be configured to detect a cavitation signal. For example, a cavitation signal can be generated when microbubbles are stimulated by FUS, and the diagnostic phase array can detect a cavitation signal. In non-limiting embodiments, the diagnostic phase transducer can include multiple elements. For example, the diagnostic phase can be a 64-element phased array transducer.

In certain embodiments, the predetermined ultrasound parameter of the diagnostic phase array transducer can include a center frequency. For example, the center frequency can range from about 20 kilohertz (kHz) to about 15 megahertz (MHz). In non-limiting embodiments, the center frequency can range from about 0.1 MHz to about 13 MHz, from about 0.1 MHz to about 10 MHz, from about 0.1 MHz to about 7 MHz, from about 0.1 MHz to about 5 MHz, from about 0.1 MHz to about 2.5 MHz, from about 0.1 MHz to about 1 MHz, from about 0.1 MHz to about 2.5 MHz, from about 0.5 MHz to about 2.5 MHz, or from about 1 MHz to about 2.5 MHz. In non-limiting embodiments, the center frequency of the diagnostic phase array transducer can be about 2.5 MHz. In non-limiting embodiments, the pulse length can range from about 5 μs to about 100 ms. In some embodiments, the pulse repetition frequency can range from about 1 Hz to about 5 kHz.

In certain embodiments, the predetermined ultrasound parameter of the diagnostic phase array transducer can include a sample rate. The diagnostic phase array transducer is configured to acquire the cavitation signal at a sample rate from about 1 MHz to about 100 MHz, from about 1 MHz to about 90 MHz, from about 1 MHz to about 80 MHz, from about 1 MHz to about 70 MHz, from about 1 MHz to about 60 MHz, from about 1 MHz to about 50 MHz, from about 1 MHz to about 40 MHz, from about 1 MHz to about 30 MHz, from about 1 MHz to about 20 MHz, from about 1 MHz to about 10 MHz, from about 1 MHz to about 5 MHz, or from about 1 MHz to about 3 MHz. In non-limiting embodiments, the sample rate is about 2.5 MHz.

In certain embodiments, the disclosed diagnostic phase array transducer can have a diameter ranging from about 10 millimeters (mm) to about 60 mm, from about 10 mm to about 50 mm, from about 10 mm to about 40 mm, from about 20 mm to about 40 mm, or from about 30 mm to about 40 mm. The focal depth of the diagnostic phase array transducer can range from about 30 millimeters (mm) to about 200 mm, from about 30 mm to about 150 mm, from about 40 mm to about 150 mm, from about 50 mm to about 150 mm, or from about 100 mm to about 150 mm.

In certain embodiments, the diagnostic phase array transducer can detect the cavitation signals to determine the types/modes of the cavitation. For example, the diagnostic phase array transducer can detect harmonic peaks, ultra-harmonic peaks, broadband emissions, cavitation magnitude, cavitation duration, and microbubble velocity to identify stable or inertial cavitation. In stable cavitation, the microbubble expands and contracts with the acoustic pressure rarefaction and compression over several cycles, and such action can result in the displacement of the vessel diameter through dilation and contraction. In inertial cavitation, the bubble can expand to several factors greater than its equilibrium radius and subsequently collapse due to the inertia of the surrounding media, thus also inducing a potential alteration of the vascular physiology. The diagnostic phase array transducer can detect the cavitation signals that can be used for calculating stable harmonic, stable ultra-harmonic and inertial cavitation levels.

In certain embodiments, the FUS transducer and the diagnostic phase array transducer can be positioned to improve the application of the FUS and the acquisition of cavitation signals. For example, the diagnostic phase array transducer can be inserted into a central opening of the FUS transducer. When the diagnostic phase array is inserted into the central opening, the FUS focus and the imaging plane can be aligned with each other, which allows for receiving the cavitation energy more efficiently.

In certain embodiments, the disclosed system can include microbubbles. The microbubbles can be configured to react to a predetermined pulse of the FUS and induce cavitation for opening the target tissue. The size of the microbubbles can range from about 1 nanometer to about 10 microns, from about 1 micron to about 9 microns, from about 1 micron to about 8 microns, from about 1 micron to about 7 microns, from about 1 micron to about 6 microns, from about 1 micron to about 6 microns, from about 1 micron to about 5 microns, from about 2 microns to about 5 microns, from about 3 microns to about 5 microns, or from about 4 microns to about 5 microns. In non-limiting embodiments, the dose of the microbubbles can be adjusted depending on the subject. For example, clinical doses (e.g., about 10 μl/kg) of microbubbles for ultrasound imaging applications can be administered to a human subject.

In certain embodiments, the disclosed system can induce the cavitation of microbubbles to open the target tissue by applying a low-pressure FUS. For example, a low-pressure FUS (e.g., about 0.1 MPa to about 1 MPa) can induce the cavitation of microbubbles to open the blood-brain barrier of a subject.

In certain embodiments, the microbubbles are configured to carry or be coated with an active agent. The microbubbles can be configured to carry an active agent (e.g., small molecule) and be acoustically activated. For example, the molecule-carrying microbubbles can carry or be coated with medicinal molecules and/or a contrast agent and/or a biomarker and/or a liposome. Medicinal molecules and/or contrast agents can also be separately positioned in proximity to the targeted region. For example, the active agent can include a monoclonal antibody, a neuronal growth factor, a chemotherapeutic agent, or a combination thereof. In some embodiments, the FUS-induced microbubble cavitation can open the target tissue without damaging the target tissue.

In certain embodiments, the target tissue can be any tissue. For example, the target tissue can be a nerve, a brain, a heart, muscle, tendons, ligaments, skin, vessels, a blood-brain barrier, a subcortical brain structure, a hippocampus, a caudate-putamen, a brain parenchyma, or a combination thereof. In non-limiting embodiments, the target tissue can be a cortical and/or a subcortical region of the brain.

In certain embodiments, the disclosed system can include a processor that can map the spatial distribution of cavitation activity using passive acoustic mapping. Passive acoustic mapping can be used to locate the activity of cavitation agents during FUS treatment. The disclosed microbubbles exposed to the FUS ultrasound can produce their own acoustic emissions, which can be passively captured by an imaging array and then processed with passive acoustic mapping algorithms. The outcome can be a passive acoustic map, which can be correlated with the spatial distribution of the induced bioeffect.

In certain embodiments, the processor can be configured to generate a cavitation map based on a spatio-temporal cavitation intensity. In non-limiting embodiments, the spatio-temporal cavitation intensity can be calculated using a coherence factor. The coherence factor can be a ratio between a coherent power to an inherent power of a channel signal acquired in a FUS bust. The coherence factor can be 1 when channel signals are coherent. The coherence factor can be zero when the channel signals are incoherent. The CF of the channel signals can be obtained for the pixel and the time. In non-limiting embodiments, the spatio-temporal cavitation intensity can be estimated by employing the CF as a weighting factor.

In certain embodiments, the processor can generate a cavitation map for each FUS burst (e.g., less than 10 ms). In non-limiting embodiments, each cavitation map can be integrated over time. The integrated map can be converted into a summation over time based on the sampling rate.

In certain embodiments, the processor can be configured to create a spatio-temporal cavitation intensity map, in which each pixel represents the beamformed cavitation intensity at a certain location and a certain time point. In non-limiting embodiments, the processor can be configured to create a cavitation map by integrating the spatio-temporal cavitation intensity over temporal pixels. For example, the processor can generate a cavitation map for each FUS burst (e.g., less than 10 ms). In non-limiting embodiments, each cavitation map can be integrated over time. The integrated map can be converted into a summation over time based on the sampling rate.

In certain embodiments, the processor can create multiple threads for parallel processing and each thread computes each pixel of the spatial-temporal map. In non-limiting embodiments, the processor can calculate the spatio-temporal cavitation intensity at a location and a time point in each thread. In non-limiting embodiments, the thread can be a single sequential flow of execution, and parallel programming can be performed by running multiple threads simultaneously. In CUDA, there can be a hierarchy for the threads (e.g., thread<thread block<grid). Multiple threads can be defined in a thread block, and multiple thread blocks can be defined within a grid. For example, the real-time CF-PAM can be implemented using a CPU or a GPU compute unified device architecture (CUDA). Threads can be created in each thread block with a grid size (e.g., [Nx/nt]×Nz×Nt, where nt is the maximum number of threads per block, Nx and Nz are the numbers of pixels of the resultant map in the lateral (x) and axial (z) directions). In non-limiting embodiments, in each thread, the cavitation intensity at a location and a time point can be obtained, and a spatio-temporal cavitation intensity map with a size of Nx×Nz×Nt can be obtained. In non-limiting embodiments, the cavitation map can be then obtained by integrating over Nt temporal pixels.

In certain embodiments, the disclosed system can compute the spatio-temporal cavitation intensity map and the number of spatio-temporal pixels can be Nx×Nz×Nt. The Nx and Nz can be the number of lateral positions (in the x direction) and the number of axial positions (in the z direction)), respectively. In non-limiting embodiments, the Nx and Nz can be the number of spatial pixels of the resultant 2D cavitation map in the lateral (x) and axial (z) directions.

In certain embodiments, the disclosed system can compute each spatio-temporal pixel in each thread. For example, when Nx, Nz, and Nt are identified (e.g., Nx=20, Nz=20, Nt=1000, and Nx×Nz×Nt=400,000), the disclosed system create 400,000 threads and assigned “a job of the calculation of one spatio-temporal pixel” to each thread, so that the total 400,000 pixels can be computed, allowing 400 different spatial locations and 1000 time points.

In certain embodiments, the disclosed system can assign multiple threads “in the temporal direction” in a thread block. Multiple threads can be in a thread block, and the maximum number of threads in a thread block can be selected. For example, if a predetermined number of threads (e.g., 250) is assigned in a thread block (nt=max number of threads per block=250), 1,600 thread blocks, each of which contains 250 threads can be used. When 250 threads are assigned to each block, a group of 250 threads, which are for the same spatial location but different time points, can be selected. This temporal-direction thread assignment can enhance the processing speed because the spatio-temporal data can be integrated over time domain afterward.

In certain embodiments, the disclosed system can include a neuronavigation system that can be configured to locate and/or monitor the target tissue. The neuronavigation system can be used for locating the transducer relative to the patient's head for the guidance of targeting. The neuronavigation system can include an infrared camera to track the position of the fiducial markers that can be attached to the transducer or patient's head. The system can show the real-time location of the FUS focus inside the brain by using the tracked positions of the transducer and the patient's head.

In certain embodiments, the disclosed system can further include a transducer tracker, a position sensor, a radiofrequency amplifier, a portable chair, and a display. The transducer and subject trackers can include infrared light-reflecting spheres and be configured to perform real-time monitoring of the position of the transducer and subject in space. The radiofrequency can amplify an amplification (e.g., 55-dB) of the signal generated by the function generator before application onto the single-element transducer.

In certain embodiments, the disclosed system can include a processor coupled to the single-element transducer and/or the navigation guidance device. In non-limiting embodiments, the processor can be coupled to the probes directly (e.g., wire connection or installation into the probes) or indirectly (e.g., wireless connection). The processor can be configured to perform the instructions specified by software stored in a hard drive, a removable storage medium, or any other storage media. The software can include computer codes, which can be written in a variety of languages, e.g., MATLAB and/or Microsoft Visual C++. Additionally, or alternatively, the processor can include hardware logic, such as logic implemented in an application-specific integrated circuit (ASIC). The processor can be configured to control one or more of the system components described above. For example, and as embodied herein, the processor can be configured to control imaging and ultrasound stimulation. The processor can be configured to control the output of the function generator and/or the transducer to provide the FUS to the subject. Additionally, or alternatively, the processor can be configured to map the cavitation activities of microbubbles.

In certain embodiments, the disclosed subject matter provides a method for passive acoustic mapping. An example method can include applying a focused ultrasound to induce a cavitation signal, receiving the cavitation signal, calculating a spatio-temporal cavitation intensity using a coherence factor, and generating a cavitation map based on the spatio-temporal cavitation intensity. In non-limiting embodiments, the cavitation signal can be a radio frequency signal. For example, the cavitation signal can be a radio frequency signal generated from microbubbles that are exposed to FUS. In non-limiting embodiments, the coherence factor can be a ratio between a coherent power to an inherent power of a channel signal acquired in a FUS bust. For example, the coherence factor can be 1 when channel signals are coherent, or the coherence factor can be zero when the channel signals are incoherent.

In certain embodiments, the FUS can be applied to stimulate microbubbles and induce cavitation signals from the microbubbles using a FUS transducer. For example, a single-element FUS transducer with a central opening (center frequency: 0.25 MHz, outer diameter: 110 mm, inner diameter: 44 mm, a radius of curvature: 110 mm, the −6 dB focal width (6 mm) and length (49 mm) can be used. A phased array imaging transducer can be used to detect the cavitation signal with predetermined parameters (e.g., center frequency: 2.5 MHz, number of elements: 64) that can be co-aligned with the FUS transducer through the central opening and can be connected to the disclosed ultrasound system. The radio-frequency (RF) data can be acquired at a sampling rate (fs) of 10.

In certain embodiments, the method can further include modifying the parameter of the FUS transducer. For example, the parameter can include a center frequency, an outer diameter, an inner diameter, a radius of curvature, or a combination thereof.

In certain embodiments, the disclosed method can further include creating a thread for each pixel of the spatial-temporal map, calculating the spatio-temporal cavitation intensity at a location and a time point in each thread, and creating a cavitation map by integrating the spatio-temporal cavitation intensity over temporal pixels. The spatio-temporal cavitation intensity can be a beamformed cavitation intensity at a certain location and a certain time point. For example, the processor can generate a cavitation map for each FUS burst (e.g., less than 10 ms). In non-limiting embodiments, each cavitation map can be integrated over time. The integrated map can be converted into a summation over time based on the sampling rate.

In certain embodiments, the disclosed subject matter can include introducing microbubbles to a target area. For example, the disclosed microbubbles can be delivered to a targeted area through various techniques (e.g., intravenous administration). In certain embodiments, the target tissue can be any tissue. For example, the target tissue can be a nerve, a brain, a heart, muscle, tendons, ligaments, skin, vessels, a blood-brain barrier, a subcortical brain structure, a hippocampus, a caudate-putamen, a brain parenchyma, or a combination thereof. In non-limiting embodiments, the target tissue can be a cortical and/or a subcortical region of the brain.

In certain embodiments, the disclosed microbubbles can be introduced to the blood-brain barrier (BBB) to open the BBB for the passage of active agents/drugs. For example, the active agent can include Definity, SonoVue, or combinations thereof. The FUS treatment with microbubbles can increase the permeability of the target tissue (e.g., BBB). In non-limiting embodiments, FUS can induce the cavitation of microbubbles to open the blood-brain barrier of a subject. The application of the FUS and microbubble can be a temporary treatment and non-invasively increase the permeability of the target tissue. During the FUS treatment with the intravenous administration of microbubbles, the cavitation map, the spectrum of the cavitation signal, and the cavitation dose can be detected after each FUS burst (e.g., at a rate of 2 Hz).

### EXAMPLES

Example 1: Real-Time Passive Acoustic Mapping With Enhanced Spatial Resolution in Neuronavigation-Guided Focused Ultrasound for Blood-Brain Barrier Opening.

The disclosed subject matter provides techniques for real-time cavitation mapping, which is capable of full-burst analysis with the enhanced spatial resolution for the neuronavigation-guided focused ultrasound (FUS) system. A diagnostic phase array transducer was inserted into the central opening of the single-element FUS transducer for cavitation signal acquisition for the stable registration between the imaging plane and the FUS beam to overcome the challenge of the imaging probe alignment. A parallel processing scheme of coherence-factor-based passive acoustic mapping (CF-PAM) was developed for enhanced spatial resolution, exploiting the lower complexity. The processing speed was evaluated by changing the number of imaging pixels and the integration time (i.e., burst length), and it was fast enough for the full burst (e.g., less than 10 ms) analysis. The spatial resolution and the processing speed of CF-PAM were compared with those of certain Time exposure acoustics (TEA) and eigenspace-based robust Capon beamformer (ERCB)-PAM techniques, which were implemented on GPU using simulation data.

The feasibility of CF-PAM for the transcranial treatment was assessed with in vitro human skull experiments and in vivo NHP experiments. In the in vivo non-human primate (NHP) model, reconstructed cavitation maps were compared with the BBB opening regions confirmed by the contrast-enhanced MRI.

Coherence-factor-based Passive Acoustic Mapping: The amplitude CF was employed for enhancing the spatial resolution of PAM. CF is the ratio between the coherent power to the incoherent power of channel signals received by multiple elements, which is equal to 1 when the channel signals are coherent and close to zero when they are incoherent. The CF of channel signals for the pixel x and the time point t is obtained by

$${C}_{j} ( x , t ) = \frac{{\vert {Σ}_{i = 1}^{{N}_{E}} \sqrt{{d}_{i} ( x )} {s}_{ij} ( t + {τ}_{i} ( x ) ) \vert}^{2}}{{N}_{E} {Σ}_{i = 1}^{{N}_{E}} {\vert \sqrt{{d}_{i} ( x )} {s}_{ij} ( t + {τ}_{i} ( x ) ) \vert}^{2}} \qquad ( 1 )$$

where Sij(t) is the acoustic signal received by the i-th element for the j-th FUS burst (j=1, 2, . . . , NB; NB: number of bursts), dᵢ(x) is the distance between the pixel x and the i-th element, and τi (x) is the round-trip time delay which consists of the transmit delay from the FUS transducer to x and the receive delay from x to the i-th element, and N_{E} is the number of transducer elements (i.e., number of channels).

The spatio-temporal cavitation intensity is estimated by employing the CF as a weighting factor as follows:

$${I}_{j} ( x , t ) = {\vert {C}_{j} ( x , t ) {Σ}_{i = 1}^{{N}_{E}} \sqrt{{d}_{i} ( x )} {s}_{ij} ( t + {τ}_{i} ( x ) ) \vert}^{2} \qquad ( 2 )$$

The cavitation map for the j-th burst is obtained by:

$${Ψ}_{j} ( x ) = {\int }_{0}^{T} {I}_{j} ( x , t ) dt \qquad ( 3 )$$

where T is the integration time which can be the burst length (i.e., 10 ms) for the full-burst integration. The integration in (3) is converted into the summation over Nₜ(=Tfₛ) when the signal sᵢⱼ(t) is sampled by a rate of fs.

GPU implementation of CF-PAM: FIG. 1(a) shows the parallel processing scheme 100 that was developed for the real-time CF-PAM. It was implemented using NVIDIA compute unified device architecture (CUDA) (Santa Clara, CA). The nt threads 101 were created in each thread block, and the grid size was [Nx/nt]×Nz×Nt 102, where nt is the maximum number of threads per block, Nx and Nz are the number of pixels of the resultant map in the lateral (x) and axial (z) directions.

In each CUDA thread, the cavitation intensity at a location and a time point were obtained. The distances di(x) and time delays τi(x) were computed, and sij(t+τi(x)) was obtained by linear interpolation between the two nearest samples; Sintp=(1−κ)·sij[k]+κ·sij[k+1] where Sintp is the interpolated value, k+κ is the time delay in samples(=τi·fs), and k and K are the integers and fractional numbers. Then, the square of the CF-weighted channel sum was computed. Receive apodization window was not applied because the effect was negligible. Since each pixel was computed at each thread, synchronization of threads was not required, which further accelerated the computation. After execution of the GPU kernel, a spatio-temporal cavitation intensity map Ij(x, t) 103 with a size of Nx×Nz×Nt was obtained. The final cavitation map 104 was then obtained by integrating over Nt temporal pixels.

To execute the CUDA code using the Verasonics sequence execution (VSX) software with MATLAB (MathWorks Inc., Natick, MA, USA), MATLAB CUDA Toolkit was used. Before the start of treatment, an initialization function was executed to allocate memory and generate a GPU kernel object using pre-compiled PTX and CUDA codes. During the treatment, immediately after each 10-ms burst, RF channel data were loaded on the global memory of the GPU, and the parallel processing was executed as an external function to run the GPU kernel for the cavitation map reconstruction. The resultant cavitation map Ψ(x, j) was updated in a graphic-user interface (GUI) after each burst to serve as a monitoring tool. The data transfer, process, and display were completed within 0.5 s before the next burst started.

GPU implementation of TEA- and ERCB-PAM: The computational speed of the proposed CF-PAM was compared with those of TEA- and ERCB-PAM. For a fair comparison, TEA and ERCB were also implemented using parallel processing. For the parallelization of ERCB-PAM, the covariance matrix calculation of delayed RF data was implemented on GPU. The calculation of weighting factors, including eigenvalue decomposition, was parallelized on a multi-core CPU instead of a GPU because it requires the eigenvalue decomposition process per pixel (i.e., per thread). The processing speed was faster when it was implemented on a multi-core CPU than on the GPU because a CPU core can be more efficient than a GPU core for complex computations such as eigenvalue decomposition. The delay-and-sum process with the ERCB weighting factors was conducted by using GPU.

FUS Sonication and Cavitation Data Acquisition: FIG. 1(b) shows the frameless FUS system with cavitation monitoring that is disclosed in this paper. A single-element FUS transducer 105 (H-231, Sonic Concepts, Bothell, WA, USA) with a central opening (center frequency: 0.25 MHz, outer diameter: 110 mm, inner diameter: 44 mm, a radius of curvature: 110 mm) was used. The −6 dB focal width and length were 6 mm and 49 mm, respectively. A phased array imaging transducer 105 (P4-2, center frequency: 2.5 MHz, number of elements: 64; ATL/Philips, Andover, MA, USA) was co-aligned with the FUS transducer through the central opening and was connected to the ultrasound system (Vantage 256, Verasonics Inc., Redmond, WA, USA). The RF data were acquired at a sampling rate (fs) of 10 MHz in all the experiments. The maximum pressure location of the focused beam relative to the imaging transducer was at x=1 mm, z=90 mm in the ultrasound image, which was found from a B-mode image of the hydrophone (HGL-0200, Onda Corp., Sunnyvale, CA, USA) when it was at the pressure maximum point.

In the in vivo experiments, a neuronavigation system (Brain-sight; Rogue Research, Montreal, QC, Canada) was used for positioning of the FUS focus at the targeted area in the brain. Since the B-mode image obtained by the array transducer and the ultrasound system can show the scalp and the skull structure in real-time, it was also used as a supplementary tool for targeting and image registration between PAM and MRI.

Once the transducer was positioned, a therapeutic pulse with a center frequency of 0.25 MHz and a pulse length of 10 ms was transmitted at a pulse repetition frequency (PRF) of 2 Hz by the FUS transducer. The cavitation data were passively detected by the imaging probe during the sonication. The acoustic signals were recorded a slightly longer than 10 ms to account for the round-trip delay.

Online Data Processing: Immediately after every burst, the RF data were transferred to the GPU memory, and a spatial cavitation map, spectrum, and doses of the cavitation signal were computed and displayed on the monitor during the treatment, as shown in FIG. 1(b). The cavitation map was computed as described in Section II.B. using CF-PAM. The stable cavitation dose with harmonic frequencies (SCDh), stable cavitation dose with ultra-harmonic frequencies (SCDu), and inertial cavitation dose (ICD) was used. The 4th to 9th harmonics and ultraharmonics were used for SCDh and SCDu, respectively, and the ICD was computed from the bandwidth of 75 kHz between the harmonic and ultraharmonic frequencies.

Simulation Setup for Spatial Resolution Evaluation: A 64-element phased array transducer with a center frequency (fc) of 2.5 MHz, and a pitch of 0.32 mm was assumed to passively receive the cavitation signal and placed at z=0 mm. A cavitation source was located at the center of the array (x=0) for the on-axis PSF or at x=10 mm for the off-axis PSF evaluations. The depth of the cavitation source (z) was changed from 50 mm to 110 mm with an interval of 10 mm. For the source separation capability assessment, two cavitation sources are placed in the axial or lateral dimension. The distance between the two sources was 40, 50, or 60 mm in the axial direction (Δz) or 3, 4, or 6 mm in the lateral direction (Δx). The signal emitted from the cavitation source was the sum of sinusoidal signals of the fundamental (fc=0.25 MHz) and the n-th harmonic (n=2, . . . , 16) frequencies with a duration of 1 ms.

The simulated RF channel data were generated using the software packages k- Wave and MATLAB. Gaussian noise was added to the RF data to have a signal-to-noise ratio of 10 dB, and the impulse response of the transducer was convoluted to get the bandlimited data assuming the −6 dB transducer bandwidth of 70% with a center frequency of 2.5 MHz. The bandlimited data were then downsampled to 10 MHz which is four times the center frequency. Data were zero-padded to take into account the initial transmit delay, which corresponds to the distance from the FUS transducer to the cavitation source.

The single cavitation source data were used to reconstruct a PSF, and the axial and lateral size of the PSF were measured by the full-width half-maximum (FWHM) of the axial and lateral profile at the peak location. Source separation capability was quantified from the PAM images with two cavitation sources. Resolvability was measured by the normalized ratio (Aratio) of peak and trough amplitudes which is Aratio=(Apeak−Atrough)/Apeak where Apeak is the peak amplitude, and Atrough is the minimum amplitude between the two sources. The peak amplitude is taken as the smaller of the two peaks.

TABLE 1
Acoustic pressure and microbubbles parameters.

| | In vitro human skull | In vivo NHP |
|---|---|---|
| Derated Pressure* | 0.1 MPa | 0.3 MPa |
| Microbubble | In-house polydisperse | Definity |
| MB concentration | 5 × 10⁸bubbles/ml | 1.2 × 10¹⁰bubbles/ml |
| MB dose | N/A | 0.05 ml/kg |

In Vitro Skull Testing Setup: FIG. 1(c) illustrates the setup for the in vitro skull experiments. To test the feasibility of the transcranial PAM, a human skull fragment 107 with a thickness range of 5.5-8.5 mm was introduced between the imaging array probe 108 and the cavitation source 109. The skull fragment 107 was placed on an ultrasonically-transparent membrane at a depth of 60 mm in the B-mode image. The skull covered the entire aperture of the imaging transducer to obtain the transcranially-detected cavitation signal.

The tube (inner diameter: 0.5 mm) was positioned within the focal area of FUS, and in-house made polydisperse microbubbles 110 were injected into the tube 111. The pressure and the concentration of the microbubbles are presented in Table I. The tube was aligned parallel to the array transducer and positioned at a depth of 90 mm with B-mode guidance. The therapeutic pulse (pulse length: 10 ms, center frequency: 0.25 MHz, PRF: 2 Hz) was used to drive the FUS transducer, and the cavitation signals from the microbubbles were collected by the imaging probe during the FUS sonication. The received data were used for the reconstruction of cavitation images with TEA-, ERCB-, and CF-PAM to evaluate the methods.

NHP Testing Setup: Experiments were performed with two rhesus macaques (NHP 1 and NHP 2; male, 6 years old), as shown in FIG. 1(b). The NHP was sedated with ketamine and atropine (10 mg/kg and 0.02-0.04 mg/kg, respectively) and maintained under anesthesia with an isoflurane-oxygen mixture. The animal was lying in a prone position on the operating table, and the transducer was placed on top of the macaque's head using a robotic arm (UR5e, Universal Robots, Denmark). The target location and trajectory of the FUS beam in the brain were planned prior to the treatment based on the T1-weighted MR image on the neuronavigation software. Positioning of the FUS focus at the planned target along the trajectory was guided by the neuronavigation system in conjunction with the B-mode imaging. The target was treated for 2 minutes with the therapeutic pulse sequence (240 bursts with a pulse length of 10 ms and a PRF of 2 Hz) with a derated pressure of 0.3 MPa, assuming the insertion loss of 0.5. At the beginning of the treatment, the microbubbles (Definity, Lantheus Medical Imaging, North Billerica, MA, USA) with a dose of 0.05 ml/kg were intravenously injected, followed by a saline flush. During the 2-min treatment, the cavitation map, spectrum, and cavitation dose plot were used for real-time monitoring.

To investigate how much the PAM signal spatially correlates with the BBB opened region, the pixel-wise correlation study was performed by a receiver operating characteristic (ROC) analysis. For the pixel-wise analysis, the PAM image and BBB opening mask were resampled with a pixel size of 0.5 mm×0.5 mm in an ROI (30 mm×40 mm) centered at the focus. Only pixels within the brain region were used for the ROC curve. The predictive capability of each pixel in the PAM image was evaluated for detecting BBB opening, and pixels in the opened region are considered positive.

MRI: Prior to the treatment, the animal with fiducial markers were scanned for anatomical registration with the neu-navigator (T1-weighted, TR/TE=7.4/3.1 ms, FA=11°, resolution=0.4×0.4×0.8 mm3; SIGNA Premier 3-T, GE Medical Systems, USA). Before and after the treatment, contrast-enhanced T1-weighted images were acquired with the same MR sequence with the gadolinium-based agent (0.2 ml/kg; Omniscan, GE Healthcare, USA) for the BBB opening confirmation and quantification. T2-weighted, susceptibility-weighted, diffusion-weighted, and apparent diffusion coefficient MRI were also obtained as safety scans to detect acute edema or hemorrhage. The scans before the treatment were used as the baseline.

For BBB opening quantification, the baseline MRI was subtracted from the post-FUS MRI, which was registered to the baseline MRI. The BBB opening volume was identified by thresholding the intensity of the subtracted MRI. The threshold was chosen such that the mean intensity of the identified volume whose pixel intensities are higher than the threshold (I_bbbo) is significantly higher than the mean intensity of background surrounding pixels (I_bkg) with a confidence level of 99% (i.e., I_{bbbo}−I_{bkg}>2.58σ_{bbbo}+bkg when ρ_{bbbo+bkg} is the standard deviation of the intensity of both regions assuming a Gaussian distribution of the pixel intensity). The MRI volume was registered with the ultrasound images (B-mode and PAM) based on the coordinates and directional vectors of the ultrasound transducer obtained from the neuronavigation system, and the B-mode image was employed to compensate for the marginal registration errors from the neuronavigation system by using the anatomical landmarks. To obtain a 2-D BBB opening map in good agreement with the 2-D PAM image, a slice with a 10-mm thickness was selected at the corresponding spatial slice in the MR volume, considering the elevational slice thickness of the array transducer for PAM. The thin volume was accumulated into a 2-D map of the BBB opening and used for the ROC study with the 2-D cavitation map.

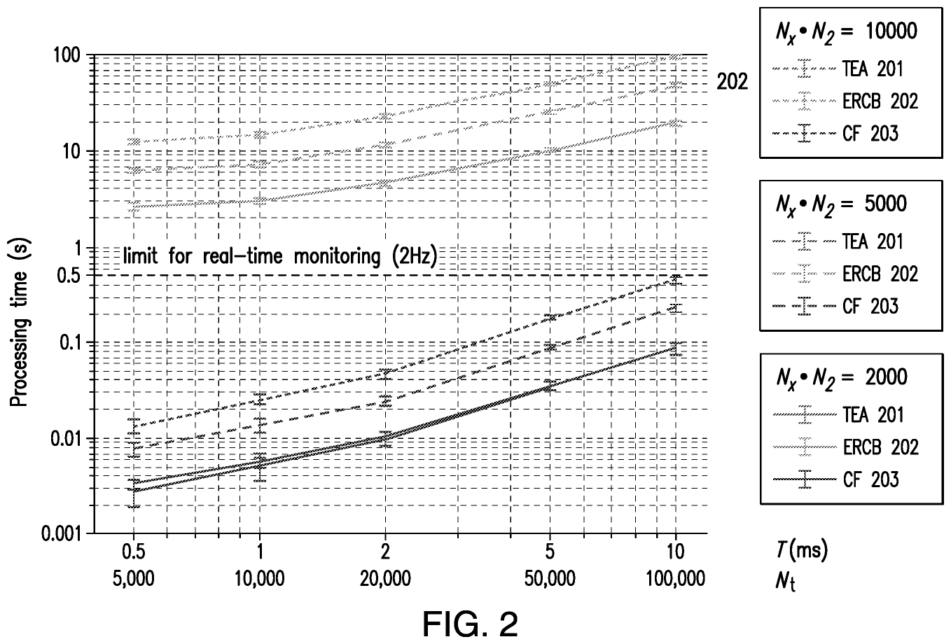

Computational Speed: All the methods were implemented on an NVIDIA Tesla V100 GPU and an Intel Xeon E5-2698 CPU for the processing speed evaluation. FIG. 2 shows the processing time for reconstruction of a single frame Ψj(x) when the integration time T is 0.5, 1, 2, 5, and 10 ms. The processing times changing the number of image pixels (Nx×Nz) from 2,000, 5000, to 10,000 was measured. The average processing time was measured from 20 repeated tests, and the error bars denote two standard deviations. Specifically, TEA 201, ERCB 202, and CF 203 required 0.22, 47.9, and 0.23 s for the reconstruction of 5,000 pixels and the temporal integration of 10 ms, respectively. ERCB-PAM took approximately 400 times longer execution time on average than those TEA-or CF-PAM, mainly due to the eigenvalue decomposition and optimization problem-solving. In contrast, the processing time required for CF-PAM was close to that of certain conventional TEA-PAM, regardless of the number of pixels and integration time. For the PRF of 2 Hz, the processing time should be less than 0.5 s and TEA-and CF-PAM were within the limit. 5,000 pixels with the full-burst integration (T=10 ms) were reconstructed using CF-PAM for real-time monitoring, taking into account the overhead time, including the sonication, data transfer, and display.

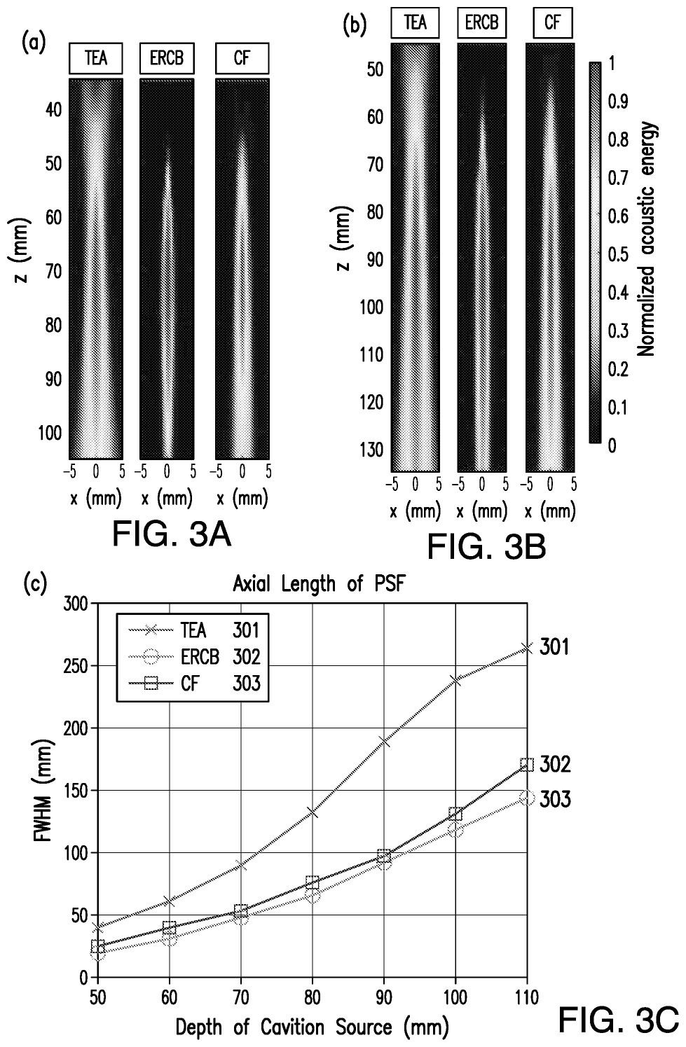

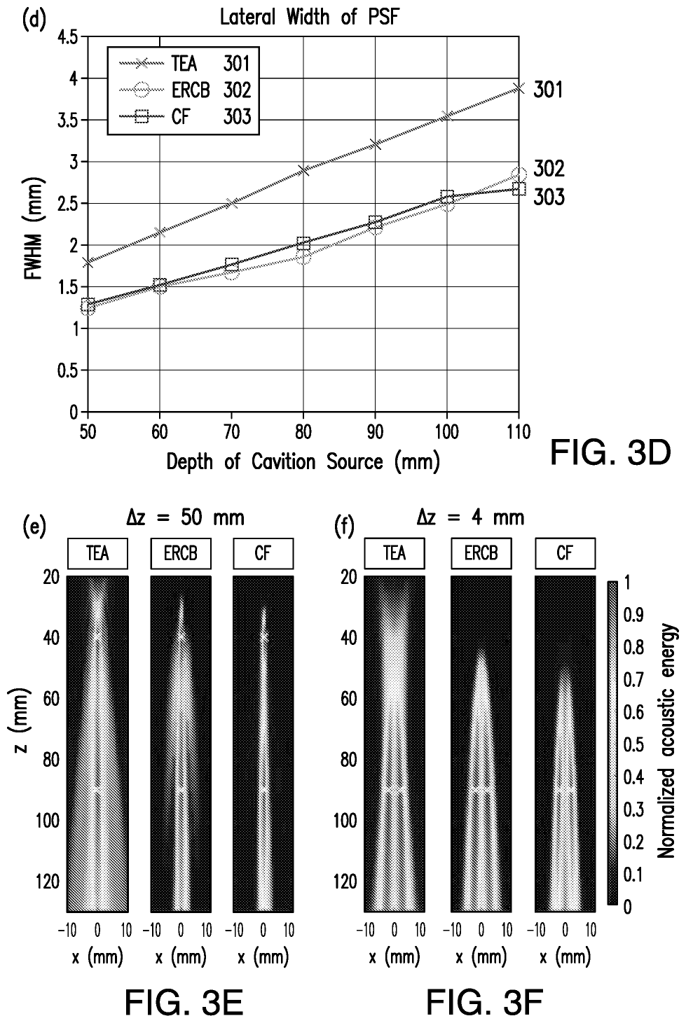

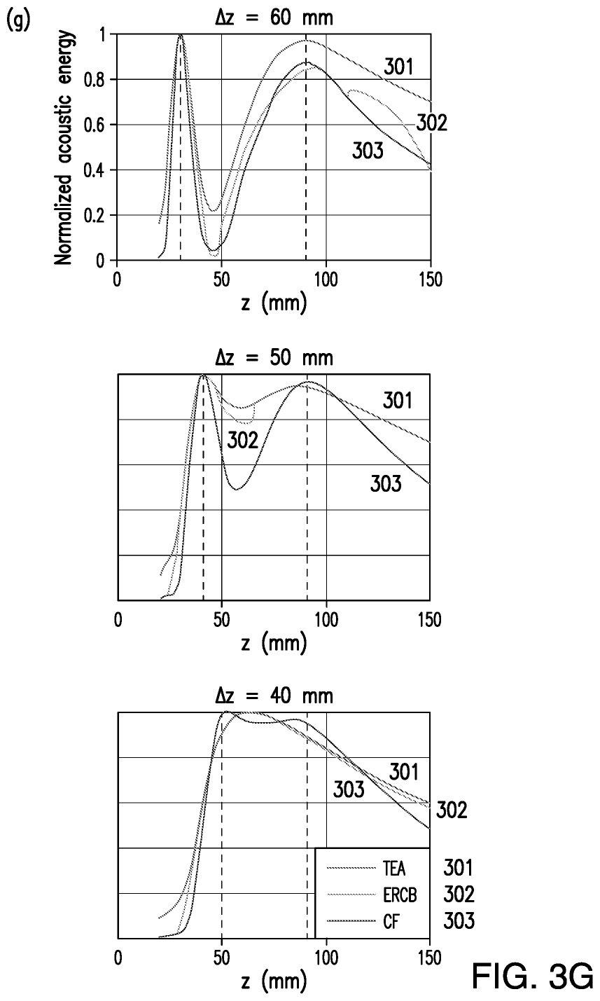

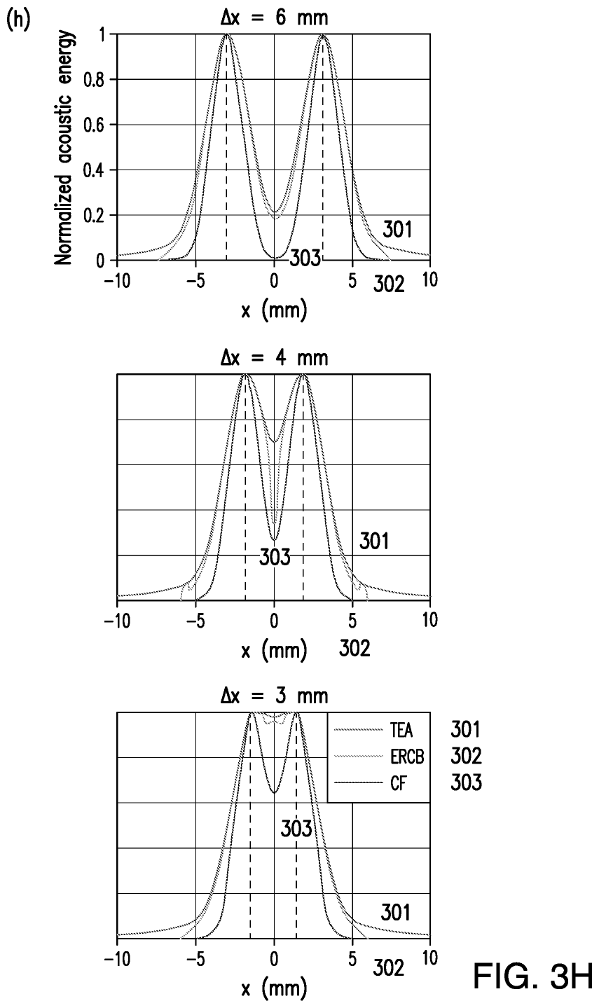

Spatial Resolution Evaluation: Using the simulation data, the PSF and the source separation capability of each method were evaluated. FIGS. 3(a)-(d) shows the PSFs and their axial length and lateral width when using TEA-PAM 301, ERCB-PAM 302, and CF-PAM 303. The PSF was elongated, and the measured FWHM increased as the depth of the cavitation source increased. Compared to the conventional TEA method, ERCB and CF methods provided smaller PSF sizes throughout all depths. The same trend was found in the off-axis PSF evaluation; the axial and lateral lengths of the PSF of the CF-PAM were similar to those of ERCB-PAM and shorter than those of CF-PAM.

Source separation capability was evaluated in the axial dimension (FIGS. 3(e) and (g)) and the lateral dimension (FIGS. 3(f) and (h)). Overall, the axial resolution was worse than the lateral resolution, mainly due to the long axial tail of PSF resulting from the small aperture of the array transducer relative to the deep imaging depth. As seen in lateral and axial profiles (FIGS. 3(g) and (h)), CF-PAM showed the best performance among the three methods for resolving two cavitation sources in both directions. The amplitude ratio of peak to the trough (Aratio) for all cases is presented in Table II. Except for the case with Δz of 60 mm, CF-PAM showed better resolvability than other methods. The parameters used in ERCB-PAM (ε=1, δ=0.5) were chosen by compromising between the spatial resolution and the distortion of the image.

TABLE II
Resolvability of two cavitation sources in axial and
lateral dimensions measured from FIGS. 3G and 3H.

| | Resolvability (Aᵣₐₜᵢₒ) | | |
|---|---|---|---|
| Δz | 60 mm | 50 mm | 40 mm |
| TEA-PAM | 0.78 | 0.1 | 0 |
| ERCB-PAM | 0.98 | 0.17 | 0 |
| CF-PAM | 0.95 | 0.5 | 0.02 |
| Δx | 6 mm | 4 mm | 3 mm |
| TEA-PAM | 0.79 | 0.3 | 0.02 |
| ERCB-PAM | 0.82 | 0.67 | 0.04 |
| CF-PAM | 0.99 | 0.73 | 0.36 |

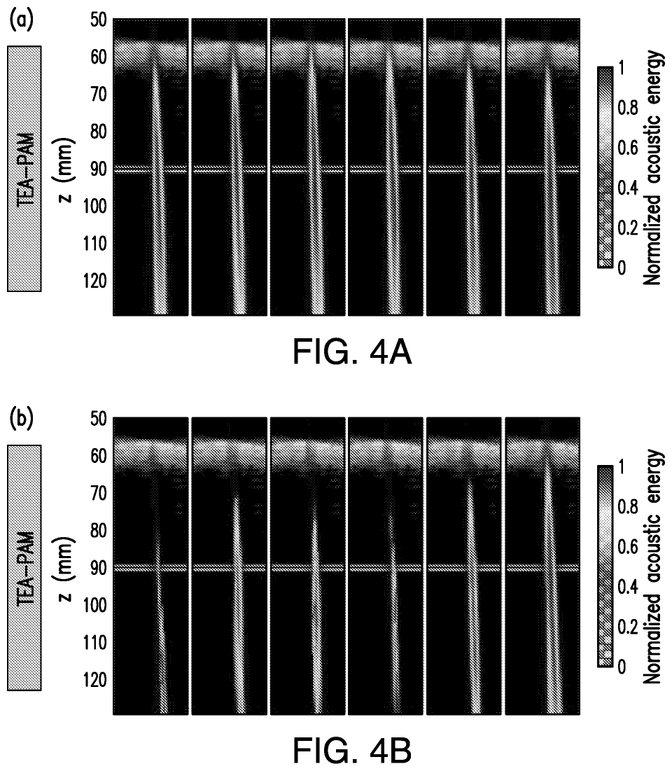

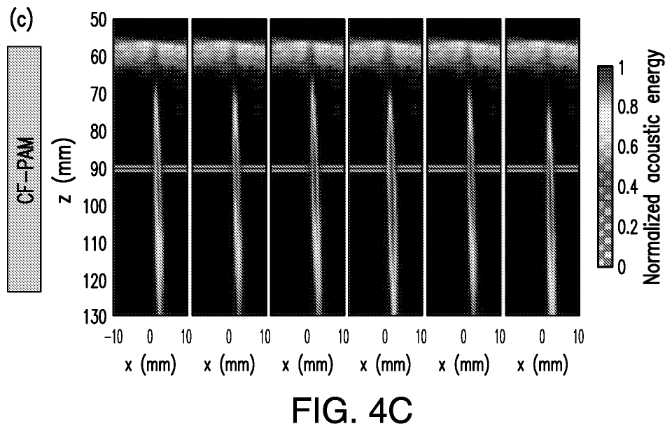

In Vitro Human Skull Studies: FIG. 4 shows the cavitation images reconstructed by using TEA-, ERCB-, and CF-PAM when the microbubbles in a tube were sonicated by FUS. The human skull fragment and the tube were positioned at z=60 mm and z=90 mm, respectively, by the guidance of a B-mode image with the imaging transducer. Microbubbles were injected into the tube, and the 10-ms therapeutic pulse was sonicated with the PRF of 2 Hz. Cavitation data from each sonication were beamformed by TEA-, ERCB-, and CF-PAM and six representative cavitation maps were shown in FIG. 4. Each cavitation map was normalized to itself.

A strong cavitation signal from the microbubble was detected around the focus (x=1 mm, z=90 mm) through the human skull with a thickness of 5.5-8.5 mm. The results show that the transcranial PAM is feasible with the phased array transducer. However, as the received cavitation data were noisy and the wavefronts were aberrated due to the skull, the spatial resolution of the cavitation map was compromised. Although ERCB-PAM (FIG. 4(b)) provided a better resolution than those of TEA-PAM (FIG. 4(a)), the cavitation signals in the ERCB-PAM maps were shifted and distorted, showing the susceptibility of the method to the noise and aberrations. On the other hand, CF-PAM provided more stable and robust cavitation maps compared to ERCB-PAM and a better spatial resolution than TEA-PAM when the skull fragment was introduced.

Out of 30 bursts, the success rate of cavitation activity localization was measured by counting the number of frames (i.e., bursts) in which the maximum acoustic energy location was within a circle centered at the focus with a radius of 5 mm. CF-PAM and TEA-PAM showed the same success rate of 0.65, while the ERCB-PAM had only 0.39. Although the success rate of the ERCB-PAM could increase with a higher & that yields less distortion, the degradation of the spatial resolution was unavoidable.

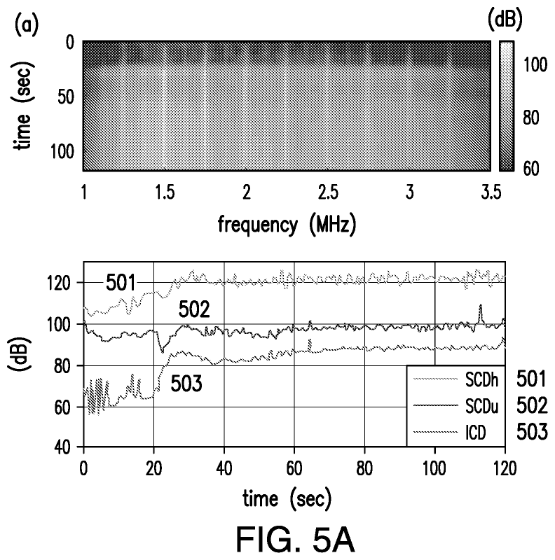

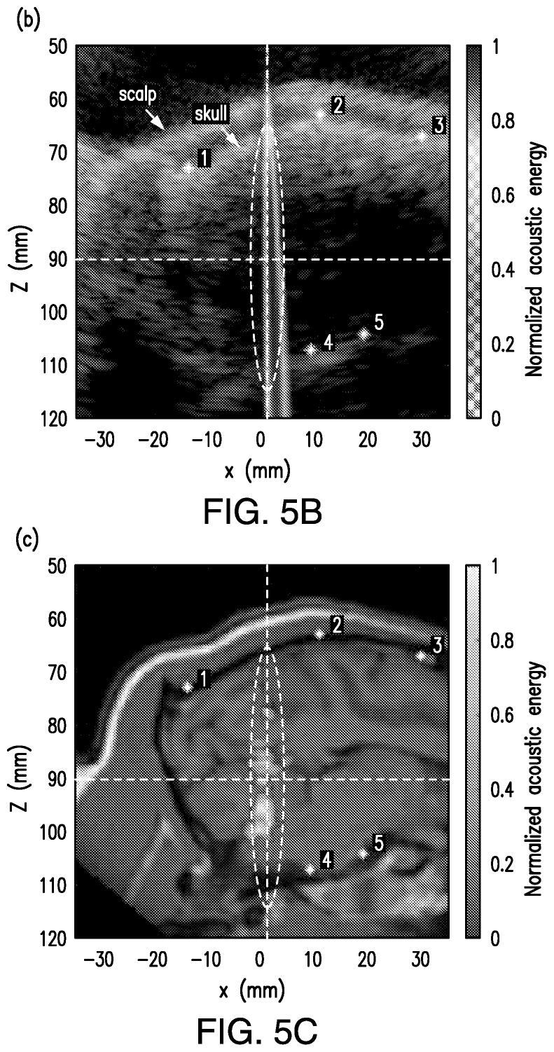

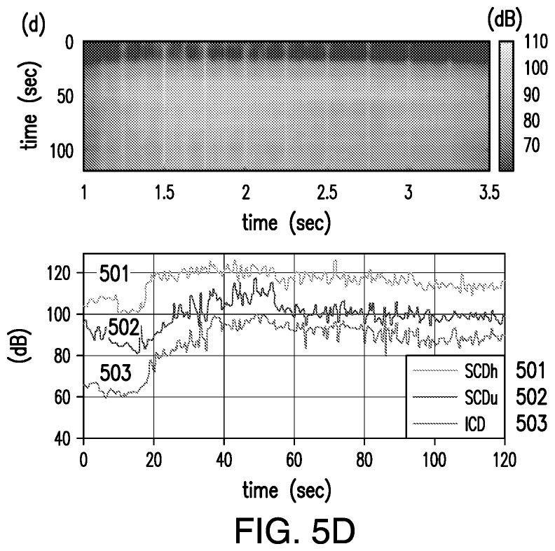

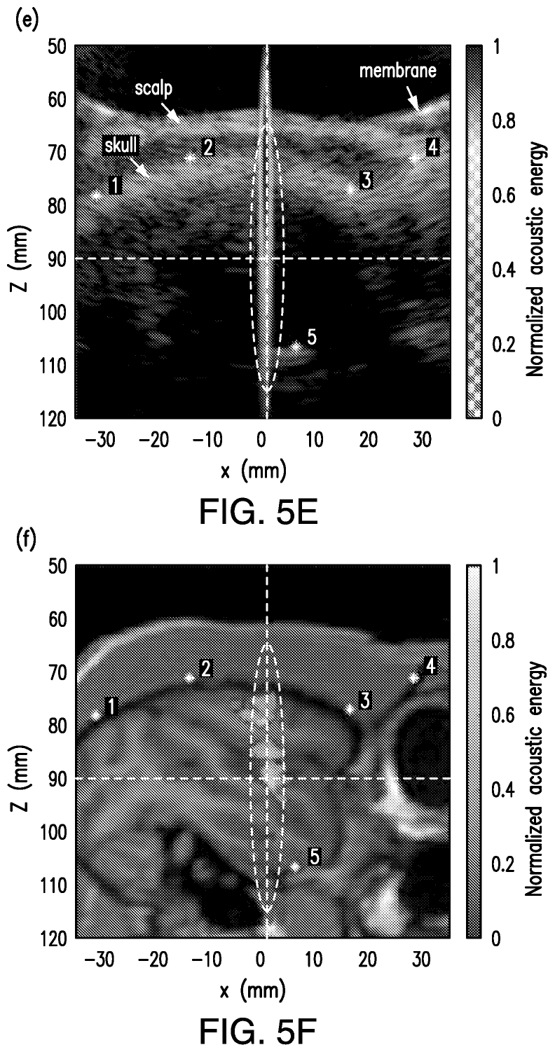

In Vivo NHP Studies: In the in vivo NHP experiments, the BBB was successfully opened without any detectable damage in the safety MR scans. FIG. 5 shows the cavitation mapping and BBB opening results in NHPs1. Spectrograms in FIGS. 5(a) and (d) show harmonic and ultraharmonic signals as well as broadband inertial cavitation over time. In the cavitation dose graphs, SCDh 501, SCDu 502, and ICD 503 increased after the microbubble injection and the saline flush (approximately at 20 s), indicating the cavitation activities from the microbubbles were distinguishable from the baseline before the injection.

In FIGS. 5(b) and (e), the grayscale B-mode images show the scalp and the skull profiles of the NHP head. The location of focus is indicated by the white-dashed lines and ellipse. The cumulative acoustic maps obtained by averaging the PAM images over all of the 240 bursts are also presented in FIGS. 5(b) and (e). Strong acoustic emissions were detected within the focal area in the brain.

The opening volume quantified from the MRI was 154 mm3 and 116 mm3 for NHP 1 and NHP 2, respectively. The ultrasound image (B-mode and PAM) and the MRI were registered. In FIGS. 5(c) and (f), the BBB opening region (color) on the slice corresponding to the PAM image slice is overlaid on the T1-weighted MRI for anatomical information. FIGS. 5(c) and (f) show that the BBB was opened within the-6 dB focal area.

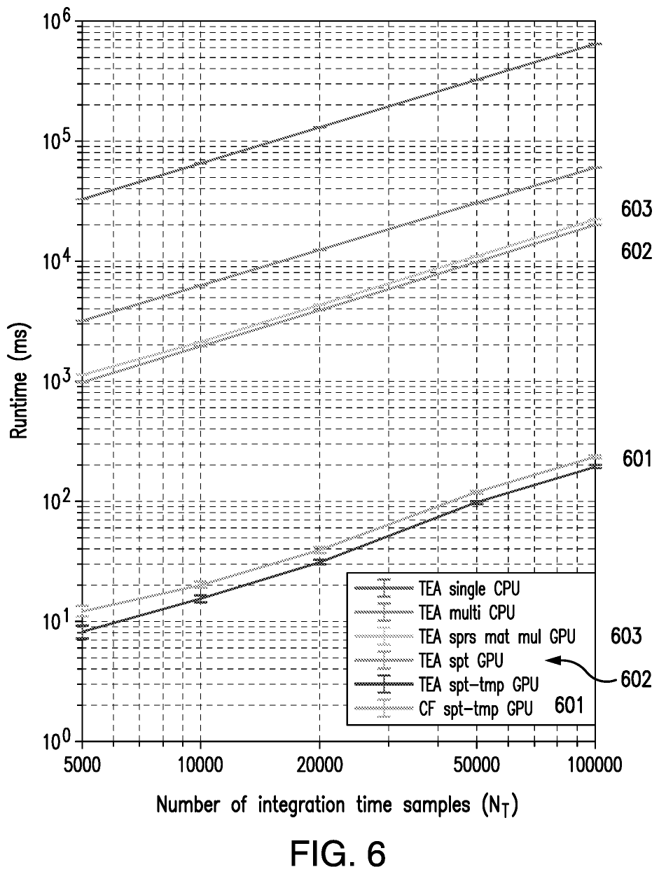

The distance between the peak location of the PAM and the centroid of the BBB opening region was 3.3 mm and 12.1 mm for NHP 1 and 2, respectively. To further evaluate the spatial correlation between the PAM and the BBB opening location, the pixel-wise ROC analysis was performed for each NHP experiment, as shown in FIG. 6. The area under the curve (AUC) was 0.824 and 0.789 for NHP 1 and 2, respectively.

Real-time Implementation of PAM with an enhanced resolution: a parallel processing scheme of CF-PAM was developed for online monitoring with a better spatial resolution than that of the conventional PAM. Its performance was evaluated and compared with ERCB-and TEA-PAM in terms of the processing speed, the size of PSF, and the resolvability of two adjacent sources. Compared with TEA-PAM, CF-PAM required a marginal overload for computing the CF as a weighting factor for each Spatio-temporal pixel (FIG. 2) while it provided a smaller PSF and a better resolution (FIG. 3). Although ERCB-PAM also yielded a smaller size of PSF and better resolution compared to TEA-PAM, the source separation capability of CF-PAM was better than that of ERCB-PAM (Table I). Compared to ERCB-PAM, CF-PAM was more stable and computationally efficient without the need for parameter optimization.

As well CF-PAM, TEA and ERCB were also implemented on GPU. While TEA-PAM was fully implemented on GPU, ERCB-PAM was conducted by a hybrid approach using a multi-core CPU and GPU. In ERCB-PAM, the weighting factors were obtained from eigenvalue decomposition for every pixel. When a large number of decompositions is required, multi-core CPU implementation can be more efficient than GPU implementation because a CPU core is more powerful than a GPU core for such a complex computation.

Therefore, the data-adaptive weighting factors were computed using a multi-core CPU, and other processing steps, including covariance matrix calculation and beamforming, were parallelized on GPU. In spite of the hybrid approach, the computational complexity of the ERCB-PAM was too high to be completed within a few seconds. A recent study reported the real-time realization of RCB-PAM, which is similar to ERCB-PAM without eigenvalue decomposition, using frequency-domain beamforming.

The proposed parallel processing scheme for PAM exploits a massive number of GPU cores by assigning each spatial-temporal pixel computation to each thread. Compared to the GPU realization, which is the disclosed computational process, the main difference of the proposed scheme herein is the on-the-fly calculation of the time delay. Since the same delay values τj (x) are used for a spatial location x across all the temporal points t (see (2) and (3)), the delay calculation for every temporal pixel can be considered redundant. For this reason, the pre-calculated delay lookup tables were used, and the sparse matrix computation method was developed for processing speed acceleration). However, the on-the-fly approach was faster than the lookup table approach due to the slow global memory speed of the GPU. Although the lookup table approach was optimized by using the shared memory, which is smaller but faster than the global memory, the overall processing speed was slower than that of the on-the-fly approach because the shared memory is allocated per thread block, and the number of threads per block is limited. In addition, the assignment of the thread block along the temporal domain was more efficient than along the spatial domain. This was because of the summation across the temporal pixels afterward.

In Vitro Human Skull Experiments: The CF-PAM was assessed and compared with other methods using a human skull fragment. The human skull effect as well to examine the feasibility of the PAM with a 2.5 MHz imaging transducer through the human skull, which is much thicker than the skull of the rhesus macaque, was assessed. The thickness of the human skull fragment used in the experiment was 5.5-8.5 mm, whereas the NHP skull thickness was 1-3 mm.

In ERCB-PAM (FIG. 4(b)), the optimization parameters (ε=50, 6=0.2) were chosen more conservatively compared to those for the simulation study (ε=1, 6=0.5) because the received cavitation signal through the skull was more noisy and aberrated. Although the smaller ε and the larger 6 provided a better spatial resolution, the adverse influence caused distortions and artifacts in the map if the values were too small or too large. For example, the success rate of cavitation activity localization reduced from 0.39 to 0.17 when the & decreased from 50 to 30. On the contrary, CF-PAM (FIG. 4(c)) showed more stable cavitation maps with enhanced spatial resolution and did not require parameter optimization.

Although a full skull cap was not used, the skull fragment was large enough to cover the incident-focused beam; the fragment size was 33×40 mm2, and the distance from the skull to the focus was 30 mm with an F-number of 1.0. In addition, all the paths from the focus to the array elements (aperture size: 20 mm) crossed the skull fragment to transcranially receive the acoustic emissions from microbubbles.

In Vivo NHP Experiments: BBB opening was performed with real-time cavitation monitoring in NHPs. Due to the low frequency (250 kHz) of the FUS for the transmission efficiency through the skull, the −6 dB focus was large (6 mm×49 mm) for NHP brains, and it included the right occipital lobe and cerebellum for NHP 1. Higher cavitation energy was detected from the deeper region (z=80−110 mm) than the near-depth region in the cumulative cavitation map (FIG. 5(b)), which agrees with the fact that BBB was opened mainly in the cerebellum (z=85−100 mm) as shown in FIG. 5(c).

For NHP 2, the focus was placed on the right putamen and hippocampus region in the frontal lobe. Within the focus, more cavitation energy was observed at shallow depths (FIG. 5 (e)), which is consistent with the BBB opening region found in the MRI (FIG. 5 (f)). Although the cumulative cavitation map (FIG. 5 (e)) showed the stronger cavitation energy at near depths (z=70-90 mm), the BBB opening region ranged from 80 mm to 100 mm at focus (FIG. 5 (f)). The distance between the peak location of the PAM and the centroid of the BBB opening was larger in NHP 2 than in NHP 1. This might be because the skull and meninges were included in the focus. The complex layers and sinus, in conjunction with the high acoustic impedance difference between the skull and the surrounding tissues, might have created reverberations of ultrasound waves and strong cavitation activities at the near-skull region.

The ROC analysis of the PAM intensity (i.e., cavitation acoustic energy) for pixel-wise binary classification of BBB opening in two NHPs is shown in FIG. 6. The AUC values were acceptable but not sufficient to be used as a confirmation tool for BBB opening. Compared to certain systems and techniques, where the cavitation map and the tissue damage showed a good correlation with high AUC, the classification performance for the BBB opening detection was relatively low. One of the reasons is the long axial length of the PSF, mainly due to the small aperture. The long tail of the PSF of PAM might have accounted for the false positives of the classification. Localization of cavitation or spatial-peak filtering could be used in the future for a better pixel-wise prediction for BBB opening.

Secondly, the cavitation threshold for BBB disruption varies in different brain tissues and different vessel types. For example, the capillaries are more susceptible to disruption than the bigger vessels, which have more endothelial cell layers than capillaries and BBB characteristics vary in different segments of the vessels. The heterogeneity of vessel size and density distribution in brain tissues may induce the different cavitation thresholds for BBB opening across the different brain tissues. Several studies also showed a higher probability of BBB opening in the gray matter than in the white matter. This spatially-variant cavitation threshold might have reduced the correlation between the cavitation energy and the BBB opening distributions. In addition, the BBB opening volume was identified by the MR signal intensity enhanced by the contrast agent, and the agent molecules can spread out into the brain parenchyma after they cross the barrier. It means that the agent can be found in a larger area than the area where cavitation activity occurred, which might have increased the false negative rate of the prediction.

In the NHP experiments, the linear phased array was positioned sagittally to have a more perpendicular incident angle to the skull. In the planning of the BBB opening procedure, the trajectory of the FUS beam was selected to have a perpendicular incident angle. However, if the array is placed coronally to the brain, the array cannot be placed parallel to the skull surface because of the huge temporalis muscle of NHP that surrounds the skull at an oblique angle. Therefore, in the procedure, once the FUS transducer was positioned at the target, the FUS transducer with the array was rotated to have a sagittal B-mode image so that the acoustic waves from the microbubbles to the array would penetrate the skull as perpendicularly as possible.

Advantages of the portable FUS system with a co-axial array transducer: The frameless and portable clinical FUS system reduced the complexity of the BBB opening treatment procedure compared to the MR-guided FUS system. This portable FUS system can successfully open the BBB with neuronavigation-system-based targeting and cavitation signal monitoring. A diagnostic linear array transducer co-axially was fixed with the FUS beam for stable alignment.

In addition, the B-mode image obtained by the array can be used for targeting in conjunction with the neuronavigation system since the disclosed linear array was co-aligned with the FUS transducer. The empirically measured registration error was 5.6±3.6 mm when using the neuronavigation system with the dental imprint platform (Rogue Research, Montreal, QC, Canada). B-mode image can show the skin and skull contours, as shown in FIGS. 5(b) and (e), and help reduce the registration errors from the neuronavigation system. After the coarse registration based on the neuronavigation system, the manual fine registration was performed by matching anatomical positions of the B-mode image and the MRI as indicated by asterisks in FIGS. 5(b), (c), (e) and (f).

Skull-Induced Acoustic Aberration Effect: Due to the complex inner structure and high sound speed of the skull, the received acoustic signals through the skull are distorted and incoherent across the channels. To compensate for the delay errors in beamforming, phase aberration correction can be performed by using experimentally-measured delay shifts or CT-based phase aberration correction. In the disclosed FUS system setup, since the array transducer has only a 20-mm aperture size, the incident angle or the time delay shift does not necessarily vary substantially across the 64 channels. However, the frequency band of the acoustic emissions used for PAM (1-3 MHz) was relatively high.

From a computational point of view, if the simulation-based or experiment-based correction factors are employed for the phase aberration correction, the use of a large lookup table is unavoidable to compensate for the delay errors of the individual channel and imaging pixel. In this case, the size of the lookup table will be NE×Nx×Nz, which can slow down the overall computation speed. On the other hand, if the effective sound speed is used for obtaining the correction factor, it can be computed and used for compensation on the fly. However, it can require reading a sound speed map in every computing thread for each pixel. As a frequency-domain beamformer, the heterogenous angular spectrum method can be a suitable option for real-time phase aberration correction.

The real-time passive cavitation mapping in the neuronavigation-guided FUS system was shown by using an imaging phased array that is co-axially aligned with the FUS transducer. By comparison studies, CF-PAM was proved to provide higher spatial resolution than TEA-PAM and more robust cavitation mapping than ERCB-PAM when the skull was introduced as an acoustic aberration. In addition, a parallel processing scheme of CF-PAM was proposed in this paper to enable real-time cavitation mapping (a PRF of 2 Hz) with the full-burst analysis (0.23 s for 5,000 imaging pixels and 100,000 temporal samples).

Transcranial cavitation mapping was also performed for in vivo BBB opening in NHPs. The cavitation map showed intermittent acoustic activity outside the focal region, but most energies were detected near the focus. The cavitation map was spatially correlated with the BBB opening region, confirmed by contrast-enhanced MRI (AUC=0.8). This study demonstrates the feasibility of real-time acoustic mapping in BBB opening with a portable FUS system for safe and efficient treatment.

FIG. 6 provides a graph showing processing times with various parallel alorithms and with various numbers of integration time samples. As shown in FIG. 6, the disclosed algorithm, spatial-temporal parallel computing (spt-tmp, 601), can be faster than other algorithms such as “spatial parallel computing (spt, 602)” and/or “sparse matrix multiplication method (sprs mat mul, 603).”

Unless otherwise defined, all technical and scientific terms used herein have the same meaning as commonly understood by one of ordinary skill in the art. In case of conflict, the present document, including definitions, will control. Certain methods and materials are described below, although methods and materials similar or equivalent to those described herein can be used in the practice or testing of the presently disclosed subject matter. All publications, patent applications, patents, and other references mentioned herein are incorporated by reference in their entirety. The materials, methods, and examples disclosed herein are illustrative only and not intended to be limiting.

While it will become apparent that the subject matter herein described is well calculated to achieve the benefits and advantages set forth above, the presently disclosed subject matter is not to be limited in scope by the specific embodiments described herein. It will be appreciated that the disclosed subject matter is susceptible to modification, variation, and change without departing from the spirit thereof. Those skilled in the art will recognize or be able to ascertain, using no more than routine experimentation, many equivalents to the specific embodiments described herein. Such equivalents are intended to be encompassed by the following claims.

What is claimed is:

1. A system for a passive acoustic mapping, comprising:
a focused ultrasound (FUS) transducer;
a diagnostic phase array transducer; wherein the diagnostic phase array transducer is configured to receive a cavitation signal induced from cavitation; and
a processor configured to generate a cavitation map based on a spatial-temporal cavitation intensity determined using a spatial-temporal parallel programming, wherein the spatial-temporal parallel programming is performed by
creating a thread for each pixel of the spatial-temporal map;
calculating the spatial-temporal cavitation intensity at a location and a time point in each thread; and
creating a cavitation map by integrating the spatial-temporal cavitation intensity over temporal pixels.

2. The system of claim 1, wherein a length of the FUS burst is less than 10 milliseconds.

3. The system of claim 1, wherein the system further comprises microbubbles, wherein the microbubbles are configured to induce the cavitation.

4. The system of claim 1, wherein the FUS transducer is a single-element FUS transducer.

5. The system of claim 1, wherein the FUS transducer has a center frequency of about 0.25 MHz.

6. The system of claim 1, wherein the diagnostic phase array transducer has a plurality of elements.

7. The system of claim 1, wherein the diagnostic phase array transducer has a center frequency of about 2.5 MHz.

8. The system of claim 1, wherein the diagnostic phase array transducer is inserted into a central opening of the FUS transducer.

9. The system of claim 1, wherein the diagnostic phase array transducer is configured to acquire the cavitation signal at a sample rate of about 10 MHz.

10. The system of claim 1, wherein the system further comprises a neuronavigation system configured to position the FUS transducer at a target area.

11. A method for passive acoustic mapping, comprising:
applying a focused ultrasound to induce a cavitation signal;
receiving the cavitation signal, wherein the cavitation signal is a radio frequency signal;
determining a spatio-temporal cavitation intensity using a spatial-temporal parallel programming, wherein the spatial-temporal parallel programming is performed by
creating a thread for each pixel of the spatial-temporal map;
calculating the spatial-temporal cavitation intensity at a location and a time point in each thread; and
creating a cavitation map by integrating the spatial-temporal cavitation intensity over temporal pixels.

12. The method of claim 11, further comprising
creating a thread for each pixel of the spatial-temporal map;
calculating the spatial-temporal cavitation intensity at a location and a time point; and
creating a cavitation map by integrating the spatial-temporal cavitation intensity over temporal pixels.

13. The method of claim 11, wherein a length of the FUS bust is less than 10 milliseconds.

14. The method of claim 11, further comprising introducing microbubbles to a target area, wherein the microbubbles are configured to induce the cavitation.

15. The method of claim 14, wherein the target area is a blood-brain barrier.

16. The method of claim 11, further comprising positioning a FUS transducer to a target area using a neuronavigation system.

17. The method of claim 16, wherein the FUS transducer is configured to apply a focused ultrasound to the target.

18. The method of claim 16, wherein the FUS transducer has a center frequency of about 0.25 MHz.

19. The method of claim 16, further comprising modifying a parameter of the FUS transducer, wherein the parameter is selected from the group consisting of a center frequency, an outer diameter, an inner diameter, a radius of curvature, and a combination thereof.
